(19) United States
(12) Patent Application Publication
(10) Pub. No.: US 2025/0152784 A1
(43) Pub. Date: May 15, 2025

(54) Fluoropolymer Based Anti-Thrombotic Coatings

(71) Applicant: CERULEAN SCIENTIFIC INC., Durham, NC (US)

(72) Inventors: Saibal BANDYOPADHYAY, Lancaster, PA (US); Andrew K. JONES, Lancaster, PA (US)

(21) Appl. No.: 19/022,651

(22) Filed: Jan. 15, 2025

Related U.S. Application Data
(63) Continuation of application No. 17/228,414, filed on Apr. 12, 2021, now Pat. No. 12,233,186.
Application No. 17/228,414 is a continuation of application No. PCT/US2019/055742, filed on Oct. 10, 2019.
(60) Provisional application No. 62/744,203, filed on Oct. 11, 2018.

(51) Int. Cl. A61L 29/08; A61M 25/00

(52) U.S. Cl. CPC A61L 29/085; A61M 25/0009

(57) ABSTRACT

The present disclosure provides for medical devices comprising one or more components comprised of a fluoropolymer and/or perfluorinated polymer and a liquid comprised of one or more fluorinated and/or perfluorinated liquids, wherein at least a portion of a surface of the one or more components comprised of a fluoropolymer and/or perfluorinated polymer comprises a surface layer of the fluorinated and/or perfluorinated liquid.

### RELATED APPLICATIONS

This application claims the benefit of U.S. Provisional Application No. 62/744,203 filed Oct. 11, 2018, which application is incorporated herein by reference in its entirety.

### BACKGROUND

When devices are inserted in the human body, they tend to induce thrombosis and attract biofilm formation. Both of these events frequently lead to infection and unintended clotting. This invention provides an inert fluorinated coating for medical devices inserted in humans which prevents the formation of biofilms and does not initiate thrombosis.

### SUMMARY

The present disclosure provides for medical devices comprising one or more components comprised of a fluoropolymer and/or perfluorinated polymer and a liquid comprised of one or more fluorinated and/or perfluorinated liquids wherein at least a portion of a surface of the one or more components comprised of a fluoropolymer and/or perfluorinated polymer comprises a surface layer of the fluorinated and/or perfluorinated liquid. Medical devices having one or more components comprised of a fluoropolymer and/or perfluorinated polymer and a liquid comprised of one or more fluorinated and/or perfluorinated liquids provided for herein include, but are not limited to, catheters, tubing, shunts, grafts, stents, extracorporeal circuits including extracorporeal membrane oxygenation circuits, implanted devices, medical testing devices, cell culture equipment (e.g., bags, plates and flasks), blood bags, and artificial organs.

The disclosure also provides for methods of making and using such medical devices in, for example, methods of treatment. In an embodiment, the methods of treatment comprise contacting mammalian (e.g., human) blood, plasma, serum, cerebrospinal fluid, and/or a mammalian (e.g., human) body or part thereof with a medical device having one or more components comprised of a fluoropolymer and/or perfluorinated polymer and a liquid comprised of one or more fluorinated and/or perfluorinated liquids.

### BRIEF DESCRIPTION OF THE DRAWINGS

FIG. 1 provides images of catheter and PICC line samples treated as in Example 1.

### DETAILED DESCRIPTION

Medical devices made with or coated with certain perfluoropolymer and/or fluoropolymers when lubricated with fluorinated and/or perfluorinated liquids show reduced thrombogenicity, biofilm adhesion, single cell adhesion and immune response. The medical devices may be prepared from a variety of perfluoropolymers and/or fluoropolymers and fluorinated and/or perfluorinated liquids and utilized in a variety of processes including medical treatment of human patients and subjects.

As used herein “fluorinated” with respect to molecules means molecules having fluorine in place of hydrogen. Fluorinated molecules include perfluorinated molecules where all hydrogens have been substituted with a fluorine.

“Fluorinated liquids” as used herein refer to chemical compositions that are liquid at 22° C. and one atmosphere of pressure comprised of, consisting essentially of, or consisting of hydrocarbons, or molecules having a hydrocarbon moiety (e.g., an alkyl group), in which one or more hydrogen atoms bound to a carbon atom have been replaced by a fluorine atom. Fluorinated liquids include perfluorinated liquids where each hydrogen atom has been replaced by a fluorine atom. The term “fluorinated liquid” is understood to include compositions comprising one or more fluorinated liquids and/or perfluorinated liquids unless stated otherwise.

Where both fluorinated liquid and perfluorinated liquid are used (e.g., fluorinated liquid and/or perfluorinated liquid), it is intended to emphasize the fact fluorinated liquids, perfluorinated liquids, and mixes of fluorinated and perfluorinated liquids can be used.

“Fluoropolymers” as used herein refer to chemical compositions comprised of, consisting essentially of, or consisting of polymers having a hydrocarbon moiety (e.g., an alkyl group), in which one or more hydrogen atoms bound to a carbon atom have been replaced by a fluorine atom. Fluoropolymers include perfluoropolymers in which each hydrogen atom has been replaced by a fluorine atom. The term “fluoropolymer” is understood to include compositions comprising one or more fluoropolymers and/or perfluoropolymers unless stated otherwise.

Where both fluoropolymers and perfluoropolymers are both used (e.g., fluoropolymers and/or perfluoropolymers), it is intended to emphasize the fact that fluoropolymers, perfluoropolymers, and mixes of fluoropolymers and perfluoropolymers can be used.

### Fluoropolymers and Forming Medical Devices

A diverse group of perfluoropolymers and/or fluoropolymers may be utilized to prepare the medical devices, or components thereof, described herein.

Among the perfluoropolymers that may be employed are: perfluoroalkoxy alkanes (PFA or PFAs when plural); polytetrafluoroethylene (PTFE); fluorinated ethylene propylene (FEP); expanded polytetrafluoroethylene (ePTFE or EPTFE); expanded fluorinated ethylene propylene (eFEP or EFEP); perfluoromethylvinylether (PMVE); perfluoro elastomers (e.g., FFKM, which are copolymers of tetrafluoroethylene and a perfluorinated ether such as PMVE sold under the tradename TECNOFLON® or TECNOFLON® PFR and branded as KALREZ®, CHEMRAZ® and PERLAST®) and combinations thereof. The fluoropolymers that may be employed include, but are not limited to, ethylene tetrafluoroethylene (ETFE); polyvinylidene fluoride (PVDF); fluoroelastomers (FKM and FEPM, sold under the tradenames VITON®, TECNOFLON®); vinylidene fluoride-hexafluoropropylene fluoroelastomer (VF2/HFP); vinylidene fluoride-hexafluoropropylene/tetrafluoro ethylene/hexafluoropropylene fluoroelastomer (VF2/tetrafluoro ethylene/HFP) terpolymer; and combinations thereof.

Medical devices, or portions thereof, may be produced from the above-mentioned fluoropolymers/perfluoropolymers by any process known in the art including, but not limited to, extrusion, co-extrusion, injection molding, compression molding, melt spinning, electrospinning, dip coating, chemical vapor deposition, blowing, foaming, and combinations thereof.

Fluorinated and/or Perfluorinated Liquids

Following production, and prior to use in a medical procedure, the medical device or a portion thereof comprising a perfluoropolymer and/or fluoropolymer is contacted with a fluorinated and/or perfluorinated liquid. The fluorinated liquid may include, but is not limited to, one or more fluorinated liquids selected from: perfluoropropane, perfluorobutane, perfluoropentane, perfluorohexane, perfluorooctane, perfluorodecalin, perfluoroperhydrophenanthrene, perfluorooctylbromide, perfluoro tributyl amine, perfluorotripentyl amine, poly(hexafluoropropylene oxide) and combinations thereof.

In an embodiment, the medical device comprises at least one component that comprises one or more perfluoropolymers and/or fluoropolymers coated (e.g., lubricated) with one or more fluorinated and/or perfluorinated liquids (e.g., perfluoropolymer lubricated with perfluorinated liquid(s)). In such an embodiment, the medical device comprises at least one component that comprises one or more perfluoropolymers coated (e.g., lubricated) with one or more perfluorinated liquids. In one such embodiment, the medical device comprises at least one component that comprises one or more perfluoropolymers coated (e.g., lubricated) with one or more fluorinated liquids. In another such embodiment, the medical device comprises at least one component that comprises one or more fluoropolymers coated (e.g., lubricated) with one or more perfluorinated liquids. In another such embodiment, the medical device comprises at least one component that comprises one or more fluoropolymers coated (e.g., lubricated) with one or more fluorinated liquids.

In an embodiment, the fluorinated and/or perfluorinated liquid used to treat (e.g., coat or lubricate) all or part of the one or more perfluoropolymer and/or fluoropolymer components of the medical device described herein comprises, consists essentially of, or consists of greater than:
(i) 50%, 60%, 70%, 80%, 90%, 95%, or 98% of one or two fluorinated and/or perfluorinated compounds on a weight basis;
(ii) 50%, 60%, 70%, 80%, 90%, 95%, or 98% fluorinated compounds on a weight basis;
(iii) 50%, 60%, 70%, 80%, 90%, 95%, or 98% perfluorinated compounds on a weight basis; or
(iv) 50%, 60%, 70%, 80%, 90%, 95%, or 98% fluorinated and/or perfluorinated compounds on a weight basis.

In an embodiment, the molar ratio of fluorine to hydrogen atoms present in the fluorinated and/or perfluorinated liquids used to treat (e.g., coat or lubricate) all or part of the one or more perfluoropolymer and/or fluoropolymer components of the medical device described herein is greater than 8.5:1, 9.0:1, 9.5:1, 9.7:1, 10:1, 15:1, 20:1, 30:1, 40:1, 50:1, 75:1, 90:1, 100:1, or 200:1 fluorine atoms:hydrogen atoms.

### Properties of the Medical Devices

Once formed, the perfluoropolymer and/or fluoropolymer surfaces of a medical device that have been treated (e.g., coated or lubricated) with fluorinated and/or perfluorinated liquid(s) (e.g., perfluoropolymer lubricated with perfluorinated liquid(s)) resist the formation, growth, or attachment of biofilms; formation or attachment of clotted blood; and/or thrombi formation induced when exposing the surface of the medical device to blood (e.g., mammalian blood such as human, porcine, ovine, bovine, canine, feline, equine, etc.).

In an embodiment, the medical device surfaces prepared from a perfluoropolymer and/or fluoropolymer and treated with fluorinated and/or perfluorinated liquids resist the growth and attachment of bacteria. The surfaces may be resistant to the growth and attachment of bacteria including, but not limited to, Actinobacillus, Acinetobacter (e.g., Acinetobacter baumannii), Aeromonas, Bordetella, Brevibacillus, Brucella, Bacteroides, Burkholderia, Borrelia, Bacillus, Campylobacter, Capnocytophaga, Cardiobacterium, Citrobacter, Clostridium, Chlamydia, Eikenella, Enterobacter, Escherichia, Francisella, Fusobacterium, Flavobacterium, Haemophilus, Helicobacter, Kingella, Klebsiella, Legionella, Listeria, Leptospirae, Moraxella, Morganella, Mycoplasma, Mycobacterium, Neisseria, Pasteurella, Proteus, Prevotella, Plesiomonas, Pseudomonas, Providencia, Rickettsia, Stenotrophomonas, Staphylococcus, Streptococcus (group A), Streptococcus agalactiae (group B), Streptococcus bovis, Streptococcus pneumoniae, Streptomyces, Salmonella, Serratia, Shigella, Spirillum, Treponema, Veillonella, Vibrio, Yersinia, Xanthomonas, and combinations thereof.

In an embodiment, the medical device surfaces prepared from a perfluoropolymer and/or fluoropolymer and treated with fluorinated and/or perfluorinated liquids resist the formation, growth, and attachment of fungi including, but not limited to, Aspergillus, Blastomyces dermatitidis, Candida, Coccidioides immitis, Cryptococcus, Histoplasma capsulatum var. capsulatum, Histoplasma capsulatum var. duboisii, Paracoccidioides brasiliensis, Sporothrix schenckii, Absidia corymbifera; Rhizomucor pusillus, Rhizopus arrhizous, and combinations thereof.

In an embodiment, the medical device surfaces prepared from a perfluoropolymer and/or fluoropolymer and treated with fluorinated and/or perfluorinated liquids resist the formation, growth, and attachment of viruses including, but not limited to, cytomegalovirus (CMV), dengue, Epstein-Barr, Hantavirus, human T-cell lymphotropic virus (HTLV I/II), Parvovirus, hepatitis, human papillomavirus (HPV), human immunodeficiency virus (HIV), acquired immunodeficiency syndrome (AIDS), respiratory syncytial virus (RSV), Varicella zoster, West Nile, herpes, polio, smallpox, yellow fever, rhinovirus, coronavirus, Orthomyxoviridae (influenza viruses), and combinations thereof.

The amount of bacteria, fungi and/or viruses associated with a surface may be measured by any means known in the art, including the total mass (e.g., the mass of bacteria or fungi), and counts of the bacteria, fungi or virus dissociated from or attached to a surface followed by counting under a microscope/electron microscope or by automated means, either of which may utilize specific labels to aid in the process. The amount/number of culturable bacteria, fungi and/or viruses bound to the surface also may be determined by growing colonies or plaques. Where the bacteria or fungi form biofilms on the medical device, the percentage of the surface area covered by the biofilm also may be used as a measure of resistance to bacterial and fungal growth and/or attachment.

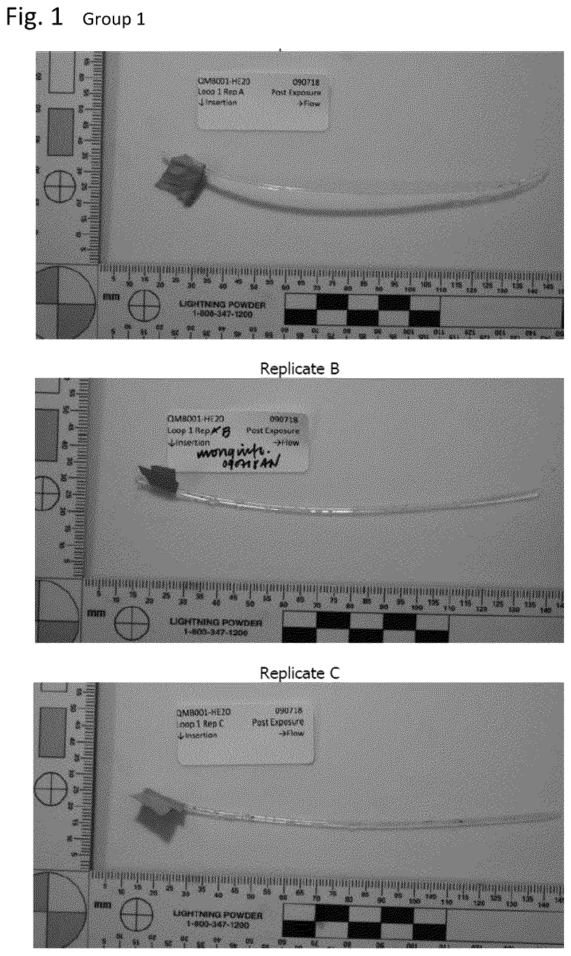

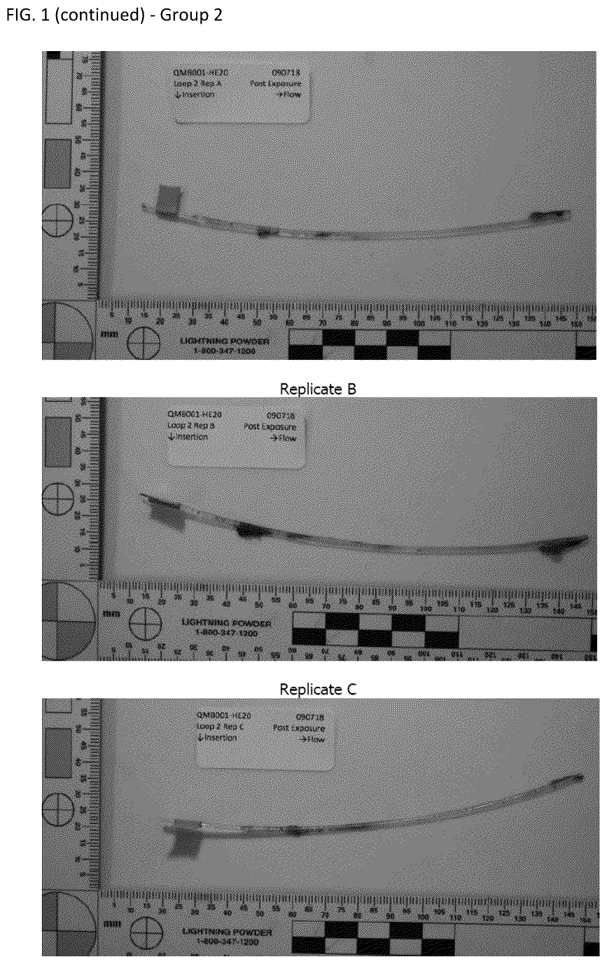

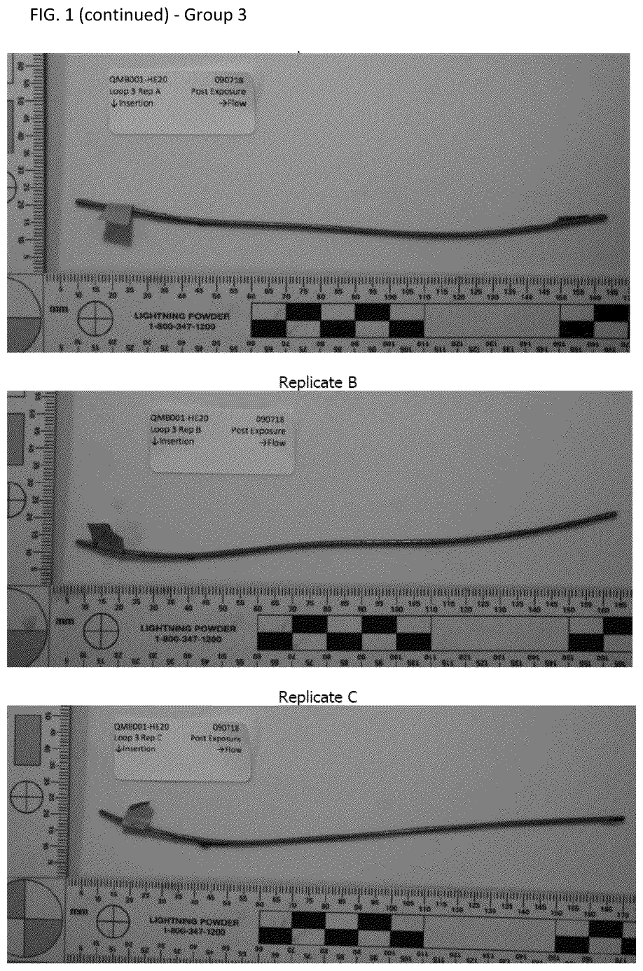

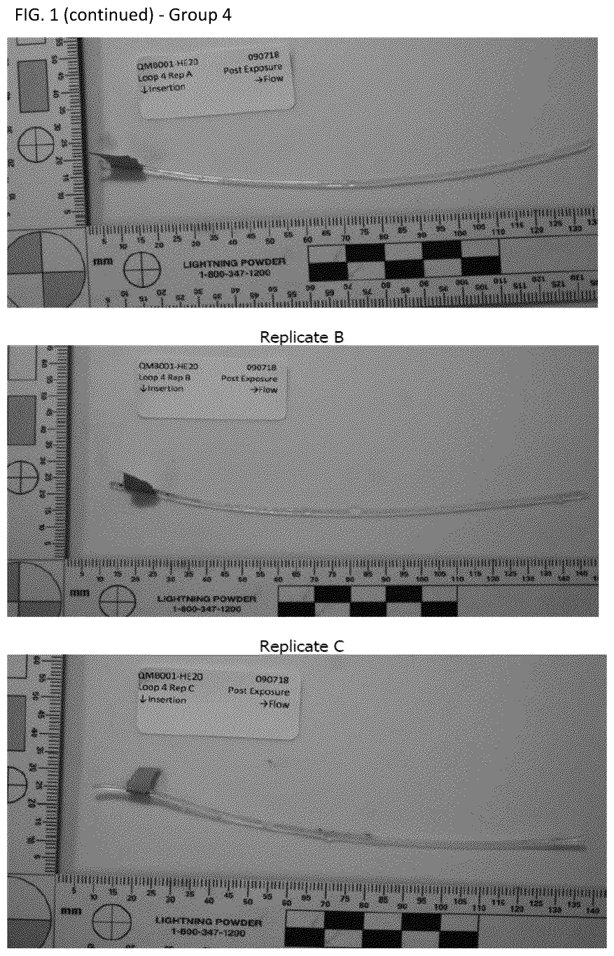

In an embodiment, the perfluoropolymer and/or fluoropolymer when lubricated with fluorinated and/or perfluorinated liquid(s) (e.g., perfluoropolymer lubricated with perfluorinated liquid(s)) is non-thrombogenic or has a reduced thrombogenicity relative to the same perfluoropolymer and/or fluoropolymer that is untreated with fluorinated and/or perfluorinated liquids. In such an embodiment, the surface of the medical device resists the formation and/or attachment of blood clots relative to the surface of the same component of the medical device prepared from the perfluoropolymer and/or fluoropolymer that has not been treated with the fluorinated and/or perfluorinated liquid(s). This may be seen in FIG. 1 comparing the catheters prepared with FEP that are treated and untreated with perfluoro decalin.

The thrombogenicity (formation or attachment of clotted blood) and/or thrombi formation associated with the perfluoropolymer and/or fluoropolymer when lubricated with fluorinated and/or perfluorinated liquid(s) (e.g., perfluoropolymer lubricated with perfluorinated liquid(s)) may be determined by any suitable method known in the art. In an embodiment, the amount of clotted blood attached to a surface may be determined by measurement of a blood specific component, such as a protein or portion of a protein, that has attached to the surface. In an embodiment, the protein may be hemoglobin. In another embodiment the protein may be fibrin (as opposed to its soluble form, fibrinogen). In another embodiment, the heme (porphyrin) group of hemoglobin may be measured (e.g., following proteolytic digestion of the adherent clot with trypsin).

In an embodiment, the fluoropolymer and/or perfluorinated polymer that comprises a layer of fluorinated and/or perfluorinated liquid resists the formation, growth, and attachment of biofilms and formation of clots and/or thrombi induced when exposing the surface of a medical device to mammalian blood. The resistance may be independent of the roughness of the perfluoropolymer and/or fluoropolymer surface (measured in the absence of any liquid), particularly where the surface is completely covered by fluorinated and/or perfluorinated liquids.

In an embodiment, at least a portion of a surface of the one or more components comprised of a fluoropolymer and/or perfluorinated polymer that comprises a layer of fluorinated and/or perfluorinated liquid has a roughness as defined by the ratio of the actual surface area divided by the projected surface area that is less than 1.01, 1.05, 1.1, 1.15, 1.25, 1.35 or 1.50, wherein the roughness is determined in the absence of the fluorinated and/or perfluorinated liquid. In such an embodiment, greater than 50%, 60%, 70%, 80%, 90%, or 95% of a surface of the one or more components comprised of a fluoropolymer and/or perfluorinated polymer that comprises a layer of fluorinated and/or perfluorinated liquid may have a roughness as defined by the ratio of the actual surface area divided by the projected surface area that is less than 1.01, 1.05, 1.1, 1.15, 1.25, 1.35 or 1.5, wherein the roughness is determined in the absence of the fluorinated and/or perfluorinated liquid.

In an embodiment, at least a portion of a surface of the one or more components comprised of a fluoropolymer and/or perfluorinated polymer that comprises a layer of fluorinated and/or perfluorinated liquid has a roughness as defined by the ratio of the actual surface area divided by the projected surface area that is greater than 1.00, 1.01, 1.05, 1.1, 1.15, 1.25, 1.35 or 1.50, wherein the roughness is determined in the absence of the fluorinated and/or perfluorinated liquid. In such an embodiment, greater than 50%, 60%, 70%, 80%, 90%, or 95% of a surface of the one or more components comprised of a fluoropolymer and/or perfluorinated polymer that comprises a layer of fluorinated and/or perfluorinated liquid may have a roughness as defined by the ratio of the actual surface area divided by the projected surface area that is greater than 1.00, 1.01, 1.05, 1.10, or 1.15, wherein the roughness is determined in the absence of the fluorinated and/or perfluorinated liquid.

In an embodiment, at least a portion of a surface of the one or more components comprised of a fluoropolymer and/or perfluorinated polymer that comprises a layer of fluorinated and/or perfluorinated liquid has a roughness as defined by the ratio of the actual surface area divided by the projected surface area that is in a range selected from 1.00 to 1.01, 1.01 to 1.05, 1.05 to 1.10, 1.10 to 1.15, 1.15 to 1.25, 1.25 to 1.35, or 1.35 to 1.50, wherein the roughness is determined in the absence of the fluorinated and/or perfluorinated liquid. In such an embodiment, greater than 50%, 60%, 70%, 80%, 90%, or 95% of a surface of the one or more components comprised of a fluoropolymer and/or perfluorinated polymer that comprises a layer of fluorinated and/or perfluorinated liquid may have a roughness as defined by the ratio of the actual surface area divided by the projected surface area that is in a range selected from 1.00 to 1.01, 1.01 to 1.05, 1.05 to 1.10, 1.10 to 1.15, or 1.15 to 1.50, wherein the roughness is determined in the absence of the fluorinated and/or perfluorinated liquid.

### CERTAIN EMBODIMENTS

1. A medical device comprising:
one or more components comprised of one or more fluoropolymers and/or perfluorinated polymers and a liquid comprised of one or more fluorinated and/or perfluorinated liquids;
wherein at least a portion of a surface of the one or more components comprised of a fluoropolymer and/or perfluorinated polymer comprises a surface layer of the fluorinated and/or perfluorinated liquid.

2. The medical device of embodiment 1, wherein the fluoropolymer and/or perfluorinated polymer are present as a coating covering at least a portion of a surface of the one or more components.

3. The medical device of embodiment 1 or embodiment 2, wherein the fluoropolymer and/or perfluoropolymer comprise one or more of: PFA, PTFE, FEP, ePTFE, eFEP, ETFE, PVDF, VF2/HFP, VF2/tetrafluoro ethylene/HFP terpolymer, fluoroelastomers and/or perfluoroelastomers.

4. The medical device of any of embodiments 1 to 3, wherein the fluoropolymer and/or perfluoropolymer comprise one or more of PFA, PTFE, FEP, ePTFE, eFEP, or ETFE.

5. The medical device of any of embodiments 1 to 3, wherein the fluoropolymer and/or perfluoropolymer comprise one or more of: PVDF, VF2/HFP, VF2/tetrafluoro ethylene/HFP terpolymer, fluoroelastomers and/or perfluoroelastomers.

6. The medical device of any of embodiments 1 to 5 wherein at least a portion of a surface of the one or more components comprised of a fluoropolymer and/or perfluorinated polymer is coated with a liquid comprising one or more fluorinated and/or perfluorinated liquid.

7. The medical device of any of embodiments 1 to 6, wherein an exterior surface comprising at least one of the one or more components comprised of a fluoropolymer and/or perfluorinated polymer is coated with a liquid comprising one or more fluorinated and/or perfluorinated liquids.

8. The medical device of any of embodiments 1 to 7, wherein greater than 50%, 75%, 80%, 90%, or 100%, of an exterior surface of at least one of the one or more components comprised of a fluoropolymer and/or perfluorinated polymer is coated with a liquid comprising one or more fluorinated and/or perfluorinated liquids.

9. The medical device of any of embodiments 1 to 8, wherein the liquid comprised of one or more fluorinated and/or perfluorinated liquids comprises, consists essentially of, or consists of one or more perfluorinated liquids.

10. The medical device of any of embodiments 1 to 8, wherein the liquid comprised of one or more fluorinated and/or perfluorinated liquid comprises greater than:
(i) 50%, 60%, 70%, 80%, 90%, 95%, or 98% of one or two fluorinated and/or perfluorinated compounds on a weight basis;
(ii) 50%, 60%, 70%, 80%, 90%, 95%, or 98% of fluorinated compounds on a weight basis;
(iii) 50%, 60%, 70%, 80%, 90%, 95%, or 98% of perfluorinated compounds on a weight basis; or
(iv) 50%, 60%, 70%, 80%, 90%, 95%, or 98% of fluorinated and/or perfluorinated compounds on a weight basis.

11. The medical device of any of embodiments 1 to 10, wherein the one or more fluorinated and/or perfluorinated liquids comprises: perfluoropropane, perfluorobutane, perfluoropentane, perfluorohexane, perfluorooctane, perfluorodecalin, perfluoroperhydrophenanthrene, perfluorooctylbromide, perfluoro tributyl amine, perfluorotripentyl amine, poly(hexafluoropropylene oxide, and combinations thereof.

12. The medical device of any of embodiments 1 to 11, wherein at least a portion of a surface of the one or more components comprised of a fluoropolymer and/or perfluorinated polymer that comprises a layer of fluorinated and/or perfluorinated liquid has a roughness as defined by the ratio of the actual surface area divided by the projected surface area that is less than 1.01, 1.05, 1.1, 1.15 or 1.5;
wherein the roughness is determined in the absence of the fluorinated and/or perfluorinated liquid.

13. The medical device of embodiment 12, wherein greater than 50%, 60%, 70%, 80%, 90%, or 95% of a surface of the one or more components comprised of a fluoropolymer and/or perfluorinated polymer that comprises a layer of fluorinated and/or perfluorinated liquid has a roughness as defined by the ratio of the actual surface area divided by the projected surface area that is less than 1.01, 1.05, 1.1, or 1.15;
wherein the roughness is determined in the absence of the fluorinated and/or perfluorinated liquid.

14. The medical device of any of embodiments 1 to 11, wherein at least a portion of a surface of the one or more components comprised of a fluoropolymer and/or perfluorinated polymer that comprises a layer of fluorinated and/or perfluorinated liquid has a roughness as defined by the ratio of the actual surface area divided by the projected surface area that is greater than 1.01, 1.05, 1.1, or 1.15; and which may be less than 1.5;
wherein the roughness is determined in the absence of the fluorinated and/or perfluorinated liquid.

15. The medical device of embodiment 14, wherein greater than 50%, 60%, 70%, 80%, 90%, or 95% of a surface of the one or more components comprised of a fluoropolymer and/or perfluorinated polymer that comprises a layer of fluorinated and/or perfluorinated liquid has a roughness as defined by the ratio of the actual surface area divided by the projected surface area that is greater than 1.00, 1.01, 1.05, 1.1, or 1.15;
wherein the roughness is determined in the absence of the fluorinated and/or perfluorinated liquid.

16. The medical device of any of embodiments 1 to 11, wherein at least a portion of a surface of the one or more components comprised of a fluoropolymer and/or perfluorinated polymer that comprises a layer of fluorinated and/or perfluorinated liquid has a roughness as defined by the ratio of the actual surface area divided by the projected surface area that is in a range selected from 1.00 to 1.01, 1.01 to 1.05, 1.05 to 1.10, 1.10 to 1.15, or 1.15 to 1.5; wherein the roughness is determined in the absence of the fluorinated and/or perfluorinated liquid.

17. The medical device of embodiment 16, wherein greater than 50%, 60%, 70%, 80%, 90%, or 95% of a surface of the one or more components comprised of a fluoropolymer and/or perfluorinated polymer that comprises a layer of fluorinated and/or perfluorinated liquid may have a roughness as defined by the ratio of the actual surface area divided by the projected surface area that is in a range selected from 1.00 to 1.01, 1.01 to 1.05, 1.05 to 1.10, 1.10 to 1.15, or 1.15 to 1.5; wherein the roughness is determined in the absence of the fluorinated and/or perfluorinated liquid.

18. The medical device of any of embodiments 1 to 17, wherein the device is selected from the group consisting of: catheters, tubing, shunts, grafts, stents, extracorporeal circuits including extracorporeal membrane oxygenation circuits, implanted devices, medical testing devices, cell culture bags, blood bags and artificial organs.

19. A method of making a medical device according to any of embodiments 1 to 18, wherein the one or more components is prepared by a method comprising one or more of: machining away excess material, extrusion, co-extrusion, injection molding, compression molding, melt spinning, electrospinning, dip coating, blowing, foaming, and chemical vapor deposition.

20. The method of embodiment 19, wherein the fluoropolymer and/or perfluorinated polymer are present as a coating on at least a portion of a surface of the one or more components, the method comprising: applying the fluoropolymer and/or perfluoropolymer by a method selected from the group consisting of: extrusion, co-extrusion, injection molding, compression molding, melt spinning, electrospinning, dip coating, blowing, foaming, and/or chemical vapor deposition.

21. A method of treatment comprising contacting mammalian blood, mammalian plasma, mammalian cerebrospinal fluid, mammalian serum, and/or a mammalian body or part thereof with a medical device according to any of embodiments 1 to 18.

22. The method of embodiment 21, wherein the medical device is contacted with blood.

23. The method of embodiment 22, wherein the blood is not heparinized or contains less than 1, 0.5, or 0.25 Howell units of heparin per ml.

24. The method of embodiment 21, wherein the medical device is contacted with a human body part.

25. The method of any of embodiments 21 to 24 wherein the device is selected from the group consisting of: catheters, tubing, shunts, grafts, stents, extracorporeal circuits including extracorporeal membrane oxygenation circuits, implanted devices, medical testing devices, cell culture bags, blood bags and artificial organs.

26. The method of treatment according to any of embodiments 21 to 25, wherein the body or part is that of a human subject or patient.

27. The method of treatment according to any of embodiments 21 to 25, wherein the treatment is conducted ex vivo or in vitro.

### EXAMPLES

### Example 1

Three catheters made from FEP, a thermal set polyurethane PICC line and a silicone catheter were obtained. Samples were, as indicated, treated with a fluorinated or perfluorinated liquid.

A sample of blood was drawn from a sheep, treated with 1 Howell unit of heparin per ml, and subsequent studies utilizing the heparinized blood were initiated within 30 minutes of the blood draw. The Activated Clotting Time (ACT) was between 150 and 250 seconds.

A test loop was prepared from 140 cm of tubing from a LivaNova® perfusion pack and filled with sheep blood heparinized as described above. Lengths of the catheters and PICC lines (between 10-15 cm) were inserted into the test loop using peel-away introducers. Each test group (type of catheter or PICC line and treatment) consisted of three replicates. For each test group a test loop was prepared and the catheter or PICC lines were inserted into the loop in the same direction with at least 5 cm between the insertion site of one device and the tip of the next device. Blood was circulated through the loop at 37±1° C. for 4 hours±30 minutes.

At the end of the 4-hour period the catheter and PICC line samples were removed, washed gently with normal saline so as not to remove blood that had clotted and attached to the samples, and the samples were photographed. Samples were scored on a scale of 1-5 as indicated in Table 1.

TABLE 1

| Thrombus | | |
|---|---|---|
| Formation | | Thrombogenicity |
| Score | Score Criterion | Evaluation |
| 0 | Minimal to nonexistent thrombus | Thromboresistant |
| | formation (1% or less) | |
| 1 | Minimal—Thrombus observed to | |
| | be covering 2 to 10% of | |
| | material length/area | |
| 2 | Mild—Thrombus observed to be | |
| | covering 11 to 25% of material | |
| | length/area | |
| 3 | Moderate—Thrombus observed to | Suspected Thrombogenic |
| | be covering 26 to 50% of | (further evaluation |
| | material length/area | recommended) |
| 4 | Extensive—Thrombus observed | Thrombogenic |
| | to be covering 51 to 75% of | |
| | material length/area | |
| 5 | Severe—Thrombus observed to | |
| | be covering >75% of material | |
| | length/area | |

Note:
Any thrombus formation associated with the insertion site for the articles should be noted and digital images prepared but is excluded from the scoring determination. This test is determining material mediated adherent thrombus formation, therefore only thrombus observed that is adhered to the article is used to determine the score.

The sample groups and final results are as follows:
Group 1: FEP treated with perfluorotributylamine—Thromboresistant
Group 2: FEP untreated—Thrombogenic
Group 3: Thermoset polyurethane PICC line untreated—Suspected Thrombogenic
Group 4: FEP treated with perfluorodecalin—Thromboresistant

1. A medical device comprising:
one or more components comprised of one or more fluoropolymers and/or perfluorinated polymers and a liquid comprised of one or more fluorinated and/or perfluorinated liquids;
wherein at least a portion of a surface of the one or more components comprised of a fluoropolymer and/or perfluorinated polymer comprises a surface layer of the fluorinated and/or perfluorinated liquid.

2. The medical device of claim 1, wherein the fluoropolymer and/or perfluorinated polymer are present as a coating covering at least a portion of a surface of the one or more components.

3. The medical device of claim 1, wherein the fluoropolymer and/or perfluoropolymer comprise one or more of: PFA, PTFE, FEP, ePTFE, eFEP, ETFE, PVDF, VF2/HFP, VF2/tetrafluoro ethylene/HFP terpolymer, fluoroelastomers and/or perfluoroelastomers.

4. The medical device of claim 3, wherein the fluoropolymer and/or perfluoropolymer comprise one or more of PFA, PTFE, FEP, ePTFE, eFEP, or ETFE.

5. The medical device of claim 3, wherein the fluoropolymer and/or perfluoropolymer comprise one or more of: PVDF, VF2/HFP, VF2/tetrafluoro ethylene/HFP terpolymer, fluoroelastomers and/or perfluoroelastomers.

6. The medical device of claim 1 wherein at least a portion of a surface of the one or more components comprised of a fluoropolymer and/or perfluorinated polymer is coated with a liquid comprising one or more fluorinated and/or perfluorinated liquid.

7. The medical device of claim 1, wherein an exterior surface comprising at least one of the one or more components comprised of a fluoropolymer and/or perfluorinated polymer is coated with a liquid comprising one or more fluorinated and/or perfluorinated liquids.

8. The medical device of claim 7, wherein greater than 50%, 75%, 80%, 90%, or 100%, of an exterior surface of at least one of the one or more components comprised of a fluoropolymer and/or perfluorinated polymer is coated with a liquid comprising one or more fluorinated and/or perfluorinated liquids.

9. The medical device of claim 1, wherein the liquid comprised of one or more fluorinated and/or perfluorinated liquids comprises one or more perfluorinated liquids.

10. The medical device of claim 1, wherein the liquid comprised of one or more fluorinated and/or perfluorinated liquids comprises greater than:
(i) 95% of one or two fluorinated and/or perfluorinated compounds on a weight basis;
(ii) 95% of fluorinated compounds on a weight basis;
(iii) 95% of perfluorinated compounds on a weight basis; or
(iv) 95% of fluorinated and/or perfluorinated compounds on a weight basis.

11. The medical device of claim 1, wherein the one or more fluorinated and/or perfluorinated liquids comprises: perfluoropropane, perfluorobutane, perfluoropentane, perfluorohexane, perfluorooctane, perfluorodecalin, perfluoroperhydrophenanthrene, perfluorooctylbromide, perfluoro tributyl amine, perfluorotripentyl amine, poly(hexafluoropropylene oxide, and combinations thereof.

12. The medical device of claim 1, wherein at least a portion of a surface of the one or more components comprised of a fluoropolymer and/or perfluorinated polymer that comprises a layer of fluorinated and/or perfluorinated liquid has a roughness as defined by the ratio of the actual surface area divided by the projected surface area that is less than 1.01, 1.05, 1.1, 1.15, or 1.5;
wherein the roughness is determined in the absence of the fluorinated and/or perfluorinated liquid.

13. The medical device of claim 12, wherein greater than 50%, 60%, 70%, 80%, 90%, or 95% of a surface of the one or more components comprised of a fluoropolymer and/or perfluorinated polymer that comprises a layer of fluorinated and/or perfluorinated liquid has a roughness as defined by the ratio of the actual surface area divided by the projected surface area that is less than 1.01, 1.05, 1.1, or 1.15;
wherein the roughness is determined in the absence of the fluorinated and/or perfluorinated liquid.

14. The medical device of claim 1, wherein at least a portion of a surface of the one or more components comprised of a fluoropolymer and/or perfluorinated polymer that comprises a layer of fluorinated and/or perfluorinated liquid has a roughness as defined by the ratio of the actual surface area divided by the projected surface area that is greater than 1.01, 1.05, 1.1, or 1.15;
wherein the roughness is determined in the absence of the fluorinated and/or perfluorinated liquid.

15. The medical device of claim 14, wherein greater than 50%, 60%, 70%, 80%, 90%, or 95% of a surface of the one or more components comprised of a fluoropolymer and/or perfluorinated polymer that comprises a layer of fluorinated and/or perfluorinated liquid has a roughness as defined by the ratio of the actual surface area divided by the projected surface area that is greater than 1.00, 1.01, 1.05, 1.1, or 1.15;
wherein the roughness is determined in the absence of the fluorinated and/or perfluorinated liquid.

16. The medical device of claim 1, wherein at least a portion of a surface of the one or more components comprised of a fluoropolymer and/or perfluorinated polymer that comprises a layer of fluorinated and/or perfluorinated liquid has a roughness as defined by the ratio of the actual surface area divided by the projected surface area that is in a range selected from 1.00 to 1.01, 1.01 to 1.05, 1.05 to 1.10, 1.10 to 1.15, or 1.15 to 1.50; wherein the roughness is determined in the absence of the fluorinated and/or perfluorinated liquid.

17. The medical device of claim 16, wherein greater than 50%, 60%, 70%, 80%, 90%, or 95% of a surface of the one or more components comprised of a fluoropolymer and/or perfluorinated polymer that comprises a layer of fluorinated and/or perfluorinated liquid may have a roughness as defined by the ratio of the actual surface area divided by the projected surface area that is in a range selected from 1.00 to 1.01, 1.01 to 1.05, 1.05 to 1.10, or 1.10 to 1.15; wherein the roughness is determined in the absence of the fluorinated and/or perfluorinated liquid.

18. The medical device of claim 1, wherein the device is selected from the group consisting of: catheters, tubing, shunts, grafts, stents, extracorporeal circuits including extracorporeal membrane oxygenation circuits, implanted devices, medical testing devices, cell culture bags, blood bags and artificial organs.

19. A method of making a medical device according to claim 18, wherein the one or more components is prepared by a method comprising one or more of: machining away excess material, extrusion, co-extrusion, injection molding, compression molding, melt spinning, electrospinning, dip coating, blowing, foaming, and chemical vapor deposition.

20. The method of claim 19, wherein the fluoropolymer and/or perfluorinated polymer are present as a coating on at least a portion of a surface of the one or more components, the method comprising: applying the fluoropolymer and/or perfluoropolymer by a method selected from the group consisting of: extrusion, co-extrusion, injection molding, compression molding, melt spinning, electrospinning, dip coating, blowing, foaming, and/or chemical vapor deposition.

21. A method of treatment comprising contacting mammalian blood, mammalian plasma, mammalian cerebrospinal fluid, mammalian serum, and/or a mammalian body or part thereof with a medical device according to claim 1.

22. The method of claim 21, wherein the medical device is contacted with blood.

23. The method of claim 22, wherein the blood is not heparinized or contains less than 1, 0.5, or 0.25 Howell units of heparin per ml.

24. The method of claim 21, wherein the medical device is contacted with a human body part.

25. The method of claim 21 wherein the device is selected from the group consisting of: catheters, tubing, shunts, grafts, stents, extracorporeal circuits including extracorporeal membrane oxygenation circuits, implanted devices, medical testing devices, cell culture bags, blood bags and artificial organs.

26. The method of treatment according to claim 21, wherein the body or part is that of a human subject or patient.

27. The method of treatment according to claim 21, wherein the treatment is conducted ex vivo or in vitro.
